(19) United States
(12) Patent Application Publication
(10) Pub. No.: US 2004/0146425 A1
(43) Pub. Date: Jul. 29, 2004

(54) Deodorizer

(76) Inventor: Ashok V. Joshi, Salt Lake City, UT (US)

Correspondence Address: TRASK BRITT, P.O. BOX 2550, SALT LAKE CITY, UT, 84110, US

(21) Appl. No.: 10/350,879

(22) Filed: Jan. 24, 2003

(51) Int. Cl. G05B 1/00; A61L 9/00

(52) U.S. Cl. 422/5; 422/123; 422/120; 422/122; 422/124; 422/3; 422/117; 422/110

(57) ABSTRACT

A device operable to form a fog or mist from a liquid oxidizing agent, and to induce that fog to contact a surface of an article or item for purpose of deodorizing that article or item by way of a chemical reaction between the oxidizing fog agent and the odiferous hydrocarbons carried on the item. Exemplary liquid oxidizing agents include aqueous solutions of hydrogen peroxide, chlorine dioxide, or bromine dioxide, and ozone containing fluids.

### TECHNICAL FIELD

The invention relates generally to apparatus operable to deodorize items, such as garments, dwellings, and vehicles. In particular, the invention applies an oxidizing agent, in a fog state, to the item being deodorized.

### BACKGROUND

It is well known that certain oxidizing agents are particularly well suited to clean and deodorize hydrocarbon-based contaminants from clothing. For example, Hydrogen peroxide is one oxidizing agent that is an ingredient in certain popular laundry detergents. However, the typical laundry cycle in which such detergent is used does not lend itself to deodorizing a wide variety of items. Certain items may be ruined by contact with water in a fluid state, or by being soaked in a fluid. Furthermore, large items, such an entire room, or an automobile, generally cannot physically be placed into a laundry machine that is generally used to launder apparel.

In certain situations, it is not desirable to launder the item, but deodorizing the item is required. One such situation maybe experienced by a traveler who soils a suit, or other wearable article, with an odoriferous contaminant. For example, a traveller may sweat while wearing a suit. Most suits require a dry cleaning laundry operation that precludes use of either the laundry detergent, or a typical laundry cycle in a clothes-laundering machine. It would often be advantageous for a traveller to avoid the inconvenience, time, and expense of dry cleaning the suit. Such a traveller would benefit from a fast-acting cleaning device conveniently operable in his room to restore the garment to an odor-free, wearable status.

Certain devices have been created to address the needs of individuals requiring refreshing and deodorizing of clothing. U.S. Pat. No. 5,815,961 discloses a clothes treating cabinet having an inflatable hanger and adapted to refresh and remove wrinkles from clothing. The entire disclosure of the '961 patent is hereby incorporated herein, as though set forth in its entirety, for its teachings of a clothes treating cabinet.

Oxidizing agents are known. Information about hydrogen peroxide, including its generation means, common uses, physical properties, and various available grades is currently published on the world wide web at the internet addresses http://www.h2o2.com and http://www.h2o2-4u.com. U.S. Pat. No. 6,479,454 is hereby incorporated herein, as though set forth in its entirety, for its teachings of antimicrobial compositions containing hydrogen peroxide.

Apparatus operable on a fluid to produce a fog state from the fluid in a fluid state are known. Certain such devices include nebulizers, atomizers, humidifiers, certain aerosol delivery systems, vaporizers, and machines with ultrasonic fluid dissipators. Angela Medical Products, of Chuangang Town, Tongzhou City, Jiangsu Province, China, has published information about commercially available nebulizers used for medical applications on the world wide web at the internet addresses http://www.nebulizer-angela.com/product1.html and http://www.nebulizer-angela.com/product2.htm. Ultranebs, of Dallas Tex., discloses information about ultrasonic nebulizers which maybe accessed on the world wide web at the internet address http://www.portablenebs.com. Aerosol delivery systems and nebulizers are disclosed by Total Care Health Industries, Inc., of New Hyde Park, N.Y., at the internet address http://tchomemedical.com/IBS/SimpleCat/Shelf/ASP/Heirarchy/04.html.

Certain devices have been adapted to produce fog from a liquid for purpose of entertainment. One such machine is known as a fog machine. Examples and descriptions of certain commercially available fog machines sold by the Audio Climax Sound & Lighting Company, of Rialto, Calif., maybe found at the internet address http://www.djtools.com/foggers.html. One fog machine and a fluid product that is commercially available and is advertised to eliminate odors, among other uses, is sold by Osburn Distributors, at the internet address http://www.nokout.com/fogmaster-jr.html.

A product advertised as an organic deodorizer is sold by the K. C. Trading Company, of Garrisonville, Va., under the name PHEW-GO. This product is to be applied to the surface to be deodorized, left to sit for 2-3 minutes, then scrubbed to remove any surface build-up. Finally, the item is to be rinsed thoroughly with fresh water. Additional information about this product can be found at the internet address http://wvw.salt-x.com.

Another product commercially available for use to deodorize items is distributed by Unsmoke-Microban Systems, of Braddock Pa. under the name Microban® Brand fine fabric deodorizer with ODORCIDE™. The fluid is to be applied to the item by spraying onto the item's surface. Additional information about this product is published on the world wide web at http://www.unsmoke.com/odor-finefabric.htm.

It would be an improvement in the art to provide an apparatus capable of applying an oxidizing agent, in a fog state, to an item to deodorize and/or sanitize that item. It would be an improvement if such apparatus were constructed for spot application of an oxidizing agent onto the item, or alternatively, constructed to treat the entire item.

### BRIEF SUMMARY OF THE INVENTION

The invention may be embodied as an apparatus for creating a deodorizing and/or sanitizing fog. A device constructed according to the invention includes a fog making device operable to convert a first fluid from a fluid state to a fog state; a circulation enhancing device operable to disperse the fog into a treatment volume; and a container configured and arranged to hold a quantity of the first fluid. The container is adapted to deliver the first fluid to the fog making device. For purposes of the invention, the first fluid is a liquid oxidizer. Liquid oxidizers within the ambit of the invention include: hydrogen peroxide, bromine dioxide, chlorine dioxide, and ozone containing solutions, as well as a plurality of such agents in combination. When hydrogen peroxide is used, the first fluid may include an aqueous solution of hydrogen peroxide in a concentration by volume of between about 1% and about 90%. Of course, potency, or efficacy of deodorizing increases as concentration of the hydrogen peroxide agent increases, but so too does the potential for creating a fog perceived as being noxious to a user. In general, the first fluid may further include a fragrant component.

The treatment volume maybe a room, or interior of an automobile, or may be defined by a structure such as a garment bag, storage container, or a cabinet. When constructed to include a cabinet, it is preferred to provide a door through which items may be loaded into the treatment volume. Certain embodiments of the invention may include a catalyst operable to neutralize fog impinging thereon and carried on an interior surface of the cabinet. Operable catalysts include noble metals and certain compounds, such as TiO₂, MnO₂, and Al₂O₃.

Preferred cabinets further include a source of warm air and structure arranged to urge the warm air to circulate within the cabinet. Furthermore, it is convenient to include control structure configurable to deliver a predetermined amount of the first fluid as a fog into the cabinet during a prescribed time interval. Preferred control structure includes electronic controls and incorporates a feedback loop to resist improper operation of the deodorizing device. Certain control systems include an indicating structure operable to indicate at least substantial dissipation of the treatment fog.

The invention can be used in a method for deodorizing garments and wearable items. The method typically includes the steps of: placing an item to be deodorized into a deodorizing cabinet; substantially sealing the cabinet to resist fluid flow therefrom; setting controls operable on one or more devices to produce a prescribed amount of fog formed from a liquid oxidizing agent and to urge the fog to contact a surface of the item; enabling operation of the one or more devices; waiting for a time interval during which said item is deodorized; and removing the item from the cabinet. The method may also include the step of, prior to enabling operation of the one or more devices; engaging a safety lock operable to resist opening of the cabinet prior to the occurrence of substantial dissipation and/or decomposition of the oxidizing fog.

Broadly, the invention maybe regarded as any apparatus arranged to create a deodorizing fog, from a fluid containing an oxidizing agent, operable to decompose and neutralize odiferous contaminants carried on an item. Typically, such contaminants are hydrocarbon-based, or sulphur-containing. Usually, a circulation enhancing device operable to disperse the fog into a treatment volume is also present. The invention includes a container adapted to deliver the first fluid to the fog making device. First fluids operable with the invention include fluid containing an oxidizer selected from the group consisting of: Hydrogen peroxide, Chlorine dioxide, Bromine dioxide, and ozone. Currently preferred embodiments of the invention include a cabinet having a door and defining the treatment volume. It is further preferred to provide a source of warm air and a device configured and arranged to urge circulation of the warm air through a volume interior to the cabinet. Additionally, it is helpful to provide an indicator device operable to indicate presence of oxidizing agent in a fog state during a treatment cycle. The deodorizing device typically includes a control system configured to operate the fog making apparatus whereby to introduce a prescribed amount of fog into the treatment volume during a first prescribed time interval, circulate warm air during a second prescribed time interval, and change the display state of the indicator device in correspondence with the presence and absence of oxidizing fog.

### BRIEF DESCRIPTION OF THE SEVERAL VIEWS OF THE DRAWINGS

FIG. 1 is a schematic illustrating a first embodiment of the invention.

FIG. 2 is a schematic illustrating a second embodiment of the invention.

### DETAILED DESCRIPTION OF THE ILLUSTRATED EMBODIMENTS

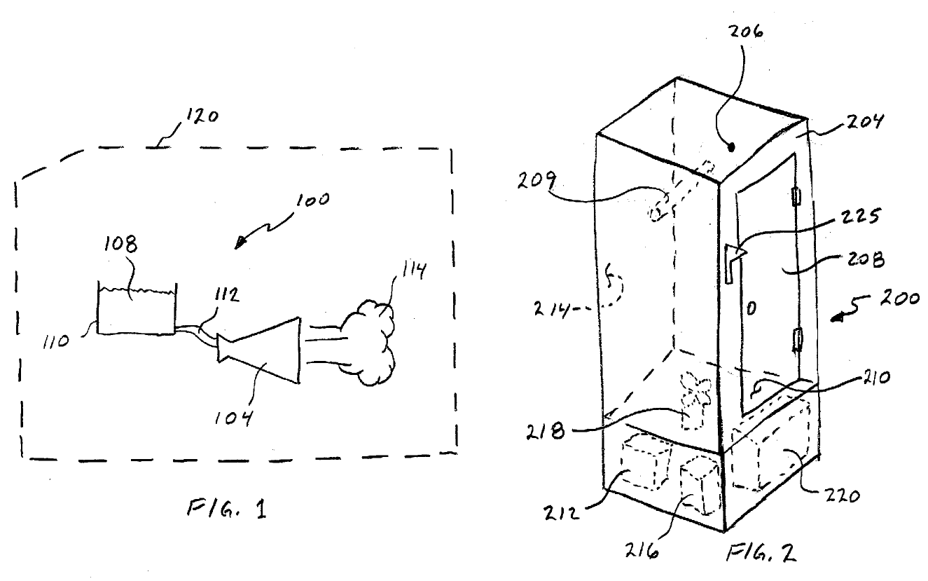

As illustrated in FIGS. 1 and 2, the invention maybe embodied as an apparatus for creating a deodorizing fog to remove odors from items including garments such as suits and dresses, and other wearable articles such as shoes, stockings, hats, and gloves. The fog is formed from a liquid oxidizing agent, and neutralizes and/or decomposes odiferous sulphur-containing or hydrocarbon-based contaminants carried on the item to be deodorized. Of course, the deodorizing capabilities of the invention maybe applied to objects other than wearable items, such as pictures, wallpaper, carpeting, furniture, drapery, upholstery, dashboards, and the like. Certain embodiments of the invention may also be applied to medical instruments and food items, as well. Typically, the deodorizing fog agent combines with undesired hydrocarbon-based or sulphur-containing contaminants to decompose those contaminants by way of a chemical reaction producing carbon dioxide or sulphur and water as end products. Certain operable oxidizing fog agents may also produce Oxygen as a reaction end product.

As illustrated in FIG. 1, a simple form of the invention, generally indicated at 100, includes a fog making device 104 operable to convert a first fluid 108 from a fluid state to a fog state. For purpose of the invention, the first fluid 108 is a liquid oxidizer. Exemplary liquid oxidizers include Hydrogen peroxide, Chlorine dioxide, Bromine dioxide, and Ozone containing fluids. In the case of Hydrogen peroxide, an aqueous solution of Hydrogen peroxide in a concentration by weight of between about 1% and about 90% can be effective. Alcohol may optionally be a portion of fluid 108. Sometimes, the fluid 108 may also include a fragrant element to impart a particular fragrance or scent to the item being deodorized. Certain desirable fragrances include floral scents, such as pine or lilac, and fruit scents, including citrus fruits such as lemon.

A fog state generally can be formed by a fluid being dispersed into a cloud of many small diameter fluid particles. Fluid particles forming a fog maybe as small as a single molecule, or as large as a visible liquid drop. However, in operation of the instant invention, it is generally preferred for the fog to be formed, at least in substantial portion, from fluid particles with sizes smaller than perceptibly discrete drops. Preferred fogs typically cause scattering of visible light, like a mist rising from a lake on a cold morning, and may therefore be visually perceived by a person operating the invention 100. Furthermore, preferred fogs can cause a feeling of general surface dampness (avoiding localized areas of wetness from discrete drops) to a person's hand if that hand were to be swept through a volume of the fog. One example of a fluid converted from a fluid state to a fog state is liquid water converted thermally to steam. A second example of a fluid converted from a fluid state to a fog state is liquid water forced through an atomizing nozzle to form a mist or fog. Operable fog making devices include nebulizers, atomizers, heat sources, ultrasonic actuators, vaporizers, aerosol delivery systems, and high pressure mechanical devices.

The invention 100 also includes a container 110 configured to hold a quantity of the liquid oxidizer 108. Operable containers 110 may be open, closed and sealed, or closeable, and can be refillable. The container 110 is further arranged, as illustrated by conduit 112, to deliver the fluid 108 to the fog making device 104. Typically, a circulation enhancing device operable to disperse the fog into a volume is included. One circulation enhancing device within contemplation is a fan.

Container 110 may also be embodied to include an electrochemical or chemical liquid oxidizer generator. For example, Hydrogen peroxide can be generated electrochemically by water electrolysis using an appropriate voltage. Similarly, Chlorine dioxide can be produced by electrolysis of a solution of sodium chlorite or sodium chlorate. Chlorine dioxide can also be produced chemically by adding a tablet containing sodium chlorate and dry acid to water. An ozone containing fluid may be produced by electrolysis of water at an appropriate voltage. Ozone may also be produced by corona discharge and the resulting gas mixed with water to form an ozone containing solution.

In certain embodiments, the fog making device 104 also can act as a fog dispersing device. An example of one such configuration is an aerosol can holding a liquid oxidizer and having an atomizing discharge nozzle capable of forming a discharged mist 114 of oxidizer particles. The invention embodied as such an aerosol-type system can be discharged in the general direction of an item to deodorized, and maybe used for spot treatment of an item. Such an aerosol-type embodiment may also be discharged into a volume 120, such as the volume of a room for deodorizing the entire room of a dwelling. Similarly, the invention may be used to deodorize the interior of a vehicle, such as a car, truck, bus, airplane, or train. The volume defined by a garment bag holding an article of clothing or other wearable item may conveniently be filled with fog from such an aerosol-type embodiment of the invention to deodorize the item.

As illustrated in FIG. 2, a second embodiment of the invention, generally indicated at 200, includes a cabinet 204 defining a treatment volume 206. One application for such a device 200 is in a hotel or motel application, where a guest may use the device 200 to deodorize a suit, or other article of clothing. Illustrated cabinet 204 includes structure having a door 208 that provides access to the treatment volume 206. Door 208 maybe closed substantially to seal the treatment volume 206 and to resist flow of air or fog from within the treatment volume 206. Items to be deodorized may be suspended from a bar 209, or placed onto cabinet floor 210. Floor 210 may include vents or ducts (not illustrated), as required. A source 212 of oxidizer agent in a fog state is conveniently located and arranged to discharge fog 114 into the volume 206. The source 212 may also include a motivator to urge the fog 114 to circulate within volume 206 of cabinet 204.

In certain embodiments of the invention 200, an interior surface 214 of the cabinet 204 may carry a catalyst operable to neutralize fog impinging thereon. Operable catalysts include noble metals and certain compounds (e.g., Ag, AgO, Ag₂O, Au, Cu, Pt, Si, SiO₂, Ti, TiO₂, and Zn.) Currently, it is preferred to apply silver, silver oxide, or silver peroxide as a coating to one or more areas on one or more interior surfaces 214 of a cabinet 204. The catalyst causes a decomposing chemical reaction with the fog agent 114 typically to form water and oxygen as reaction end products. Certain desirable coatings promote decomposition of the oxidizing agent fog 114, and also resist propagation of bacteria. One advantage provided by a catalytic surface is the more rapid decomposition of excess fog agent 114 subsequent to a treatment cycle. Therefore a user may gain access more rapidly to a deodorized item without being exposed to an oxidizing fog.

The invention 200 may further include a source of warm air 216 and structure, such as fan 218, arranged to urge the warm air to circulate within the cabinet 204. Certain desired embodiments of invention 200 further include control structure 220 configurable to deliver a predetermined amount of the first fluid 108 as a fog 114 into the cabinet 204 during a prescribed time interval. Control structure 220 maybe adjusted to operate the device 200 through an automatic deodorizing cycle, including warming and drying the items.

Preferred embodiments 200 will include some sort of indicating structure 225 operable to indicate the occurrence of at least substantial dissipation of the treatment fog 114. Indicating structure 225 may include devices such as illustrated flag 225, or simply the fog 114 itself. In the latter case, door 208 maybe manufactured to have a transparent area to provide direct observation of the treatment process and fog 114. When the fog 114 is dissipated, or decomposed, a user can remove the deodorized items without worry of encountering any potentially noxious treatment agent.

It is currently preferred for an embodiment 200 to include controlling structure, such as electronic controls 220, having a feedback loop to resist improper operation of the device 200. For example, controls 220 maybe adjusted to deliver treatment fog 114 into volume 206 for a period of 10 minutes, then to circulate warm air for 15 minutes, before changing the position of indicator flag 225 to show completion of the treatment cycle. It is within contemplation for the control 220 to effect a safety lock to resist opening of door 208 before treatment fog 114 is sufficiently dissipated.

Use of the embodiment 200 typically includes placing an item to be deodorized into a deodorizing cabinet 204 and closing the door 208 to substantially seal the cabinet 204 to resist fluid flow therefrom. A user then adjusts controls 220 operable on one or more devices (e.g. fog source 212, warm air source 216, fan 218, and indicator 225) to produce a prescribed amount of fog 114 formed from a liquid oxidizing agent 108 and to urge the fog 114 to contact a surface of the item. The user then enables operation of the one or more devices to effect a deodorizing cycle. After waiting for a time interval during which the item is deodorized, the item maybe removed from the cabinet 204. A deodorizing cycle may also include, prior to enabling operation of the one or more devices, engaging a safety lock operable to resist opening of the cabinet 204 prior to the occurrence of substantial dissipation of the fog 114.

What is claimed is:

1. An apparatus for deodorizing articles, said apparatus comprising:
a fog making device operable to convert a first fluid from a fluid state to a fog state;
a circulation enhancing device operable to disperse said fog into a volume; and
a container configured and arranged to hold a quantity of said first fluid, said container being adapted to deliver said first fluid to said fog making device; wherein:
said first fluid comprises a liquid oxidizer.

2. The apparatus of claim 1, further comprising a cabinet defining said volume.

3. The apparatus of claim 1, wherein said liquid oxidizer comprises hydrogen peroxide.

4. The apparatus of claim 3, said first fluid comprising an aqueous solution of hydrogen peroxide in a concentration by weight of between about 1% and about 40%.

5. The apparatus of claim 1, wherein said first fluid comprises an ozone containing solution.

6. The apparatus of claim 1, wherein said first fluid comprises Chlorine.

7. The apparatus of claim 2, wherein said cabinet is in fluid communication with a generator of said first fluid.

8. The apparatus of claim 1, wherein said first fluid further comprises a fragrant component.

9. The apparatus of claim 2, said cabinet comprising a garment bag.

10. The apparatus of claim 2, wherein said cabinet comprises a structure having a door.

11. The apparatus of claim 2, wherein an interior surface of said cabinet carries a catalyst operable to neutralize fog impinging thereon.

12. The apparatus of claim 11, said catalyst comprising a noble metal.

13. The apparatus of claim 10, further comprising a source of warm air and structure arranged to urge said warm air to circulate within said cabinet.

14. The apparatus of claim 10, further comprising control structure configurable to deliver a predetermined amount of said first fluid as a fog into said cabinet during a prescribed time interval.

15. The apparatus of claim 14, said control structure comprising electronic controls comprising a feedback loop to resist improper operation of said apparatus.

16. The apparatus of claim 14, further comprising an indicating structure operable to indicate at least substantial dissipation of said fog.

17. A method for deodorizing garments and wearable items, comprising the steps of:
placing an item to be deodorized into a deodorizing cabinet;
substantially sealing said cabinet to resist fluid flow therefrom;
setting controls operable on one or more devices to produce a prescribed amount of fog formed from a liquid oxidizing agent and to urge said fog to contact a surface of said item;
enabling operation of said one or more devices;
waiting for a time interval during which said item is deodorized;
removing said item from said cabinet.

18. The method of claim 17, further comprising the step of:
prior to enabling operation of said one or more devices, engaging a safety lock operable to resist opening of said cabinet prior to the occurrence of substantial dissipation of said fog.

19. An apparatus for creating a deodorizing fog, comprising:
a fog making device operable to convert a first fluid from a fluid state to a fog state;
a circulation enhancing device operable to disperse said fog into a treatment volume;
a container configured and arranged to hold a quantity of said first fluid, said container being adapted to deliver said first fluid to said fog making device; wherein:
said first fluid comprises an oxidizer selected from the group consisting of: hydrogen peroxide, chlorine dioxide, bromine dioxide, and ozone.

20. The apparatus of claim 19, further comprising:
a cabinet comprising a door and defining said volume;
a source of warm air and a device configured and arranged to urge circulation of said warm air through said volume;
an indicator device operable to indicate presence of oxidizing agent in a fog state during a treatment cycle; and
a control system configured to operate said fog making apparatus whereby to introduce a prescribed amount of fog into said volume during a first prescribed time interval, to circulate warm air during a second prescribed time interval, and to change the display state of said indicator device in correspondence with the presence and absence of fog.
